(19) United States
(12) Patent Application Publication
(10) Pub. No.: US 2001/0025028 A1
(43) Pub. Date: Sep. 27, 2001

(54) Methods of inhibiting bone resorption

(75) Inventors: John E. Fisher, Jenkintown, PA (US); Gideon A. Rodan, Bryn Mawr, PA (US)

Correspondence Address: Merck & Co., Inc., Patent Department, P.O. Box 2000 - RY60-30, Rahway, NJ, 07065-0907, US

(73) Assignee: Merck & Co., Inc.

(21) Appl. No.: 09/833,185

(22) Filed: Apr. 11, 2001

Related U.S. Application Data
(63) Continuation of application No. 09/264,730, filed on Mar. 9, 1999, now abandoned.
(60) Provisional application No. 60/092,918, filed on Jul. 15, 1998.
(60) Provisional application No. 60/078,157, filed on Mar. 16, 1998.
(60) Provisional application No. 60/077,913, filed on Mar. 13, 1998.

(51) Int. Cl. A61K 38/22; A61K 31/401; A61K 31/366; A61K 31/215

(52) U.S. Cl. 514/21; 514/423; 514/460; 514/547

(57) ABSTRACT

The present invention relates to methods of inhibiting bone resorption comprising administering a therapeutically effective amount of a 3-hydroxy-3-methylglutaryl coenzyme A reductase inhibitor.

### CROSS REFERENCE TO RELATED APPLICATIONS

The present invention is related to U.S. provisional applications Serial Nos. 60/077,913, filed Mar. 13, 1998, 60/078,157, filed Mar. 16, 1998, and 60/092,918, filed Jul. 15, 1998, the contents of which are hereby incorporated by reference.

### BRIEF DESCRIPTION OF THE INVENTION

The present invention relates to methods of inhibiting abnormal bone resorption comprising administering a therapeutically effective amount of a 3-hydroxy-3-methylglutaryl coenzyme A reductase inhibitor (hereafter “HMG-CoA reductase inhibitor”) to a mammal in need thereof. More particularly, the present invention relates to methods of treating or preventing conditions or disease states involving abnormal bone resorption by administering a therapeutically effective amount of a HMG-CoA reductase inhibitor.

### BACKGROUND OF THE INVENTION

A variety of conditions or disease states in humans and other mammals involve or are associated with abnormal bone resorption. Such disorders include, but are not limited to, osteoporosis, Paget's disease, periprosthetic bone loss or osteolysis, hypercalcemia of malignancy, osteogenesis imperfecta, osteoarthritis, and aveolar bone loss associated with periodontal disease. Furthermore, abnormal bone resorption is often an undesired side effect associated with immunosuppresive therapy and chronic glucocorticoid use. The most widespread of the bone resorption disorders is osteoporosis, which in its most frequent manifestation occurs in postmenopausal women. Osteoporosis is a systemic skeletal disease characterized by a low bone mass and microarchitectural deterioration of bone tissue, with a consequent increase in bone fragility and susceptibility to fracture. Because osteoporosis, as well as other disorders associated with abnormal bone resorption, are generally chronic conditions, it is believed that appropriate therapy will generally require chronic treatment.

Multinucleated cells called osteoclasts are responsible for causing bone loss through a process known as bone resorption. Osteoclasts are actively motile cells that migrate along the surface of bone, and that can bind to bone and secrete acids and proteases causing a resorption of mineralized bone tissue.

Therapeutic agents that have been used to treat abnormal bone resorption, and osteoporosis in particular, include organic bisphosphonates, estrogens, calcium supplements, the peptide hormone calcitonin, and sodium fluoride. See Riggs et al., The New England J. of Med., Vol. 327, No. 9, pp. 620-627, 1992, which is incorporated by reference herein in its entirety.

It is well known that bisphosphonates are selective inhibitors of osteoclastic bone resorption. The bisphosphonates are important therapeutic agents in the treatment or prevention of a variety of generalized or localized bone disorders caused by or associated with abnormal bone resorption. See H. Fleisch, Bisphosphonates In Bone Disease, From The Laboratory To The Patient, 3rd Edition, Parthenon Publishing (1997); U.S. Pat. No. 4,621,077, to Rosini et al., issued Nov. 4, 1986; U.S. Pat. No. 4,922,007, to Kieczykowski et al., issued May 1, 1990; U.S. Pat. No. 5,019, 651, to Kieczykowski et al, issued May 28, 1991; U.S. Pat. No. 5,510,517, to Dauer et al., issued Apr. 23, 1996; and U.S. Pat. No. 5,648,491, to Dauer et al., issued Jul. 15, 1997; which are all incorporated by reference herein in their entirety.

Despite their therapeutic benefits, bisphosphonates are not well absorbed from the gastrointestinal tract. See B. J. Gertz et al., Clinical Pharmacology of Alendronate Sodium, Osteoporosis Int., Suppl. 3: S13-16 (1993) and B. J. Gertz et al., Studies of the oral bioavailability of alendronate, Clinical Pharmacology & Therapeutics, vol. 58, number 3, pp. 288-298 (September 1995), which are both incorporated by reference herein in their entirety. Intravenous administration has been used to overcome this bioavailability problem. However, intravenous administration is costly and inconvenient, especially when the patient must be given an intravenous infusion lasting several hours on repeated occasions.

If oral administration of the bisphosphonate is desired, relatively high doses must be administered to compensate for the low bioavailability from the gastrointestinal tract. To offset the limited bioavailability, it is generally recommended that the patient take the bisphosphonate on an empty stomach and fast for at least 30 minutes after dosing. However, many patients find the need for such fasting on a daily basis to be inconvenient. Moreover, oral administration has been associated with adverse gastrointestinal effects, especially those relating to the esophagus. See Fleisch, Id. These effects appear to be related to the irritant potential of the bisphosphonate in the esophagus, a problem which is exacerbated by the presence of refluxed gastric acid. For example, the bisphosphonate, pamidronate has been associated with esophageal ulcers. See E. G. Lufkin et al., Pamidronate: An Unrecognized Problem in Gastrointestinal Tolerability, Osteoporosis International, 4: 320-322 (1994), which is incorporated by reference herein in its entirety.

The other above-mentioned anti-bone resorptive therapies also have disadvantages associated with them. Hormone replacement therapy, which involves the administration of estrogen and other compounds having estrogenic activity, is often prescribed for the treatment of osteoporosis in postmenopausal women. However, such therapy has disadvantages including an increased risk of certain cancers, such as breast cancer, and the development of deep vein thromboses. Also, hormone replacement therapy is contraindicated in premonopausal women and male patients.

It has been a long-held belief that the administration of calcium supplements can retard the effects of accelerated bone resorption associated with osteoporosis. However, the benefits, if any, of calcium supplementation alone are relatively small and have yet to be fully demonstrated.

The peptide hormone calcitonin is also currently used in the treatment of postmenopausal osteoporosis. However, this hormone has a relatively high molecular weight and has the disadvantage of requiring parenteral or intranasal administration. Also, many patients on calcitonin therapy develop resistance to the material associated with increased titers of antibodies that neutralize the effectiveness of the therapy.

Although sodium fluoride has been used to stimulate bone formation in osteoporotic women, the resulting bone often has an abnormal fluoride content resulting in structural defects and increased fragility.

Therefore, even though a number of different agents are known for treating abnormal bone resorption, it is seen that a need clearly exists for finding new therapeutic agents.

It has been reported that perturbation of the cholesterol biosynthetic pathway can have an effect on in vitro osteoclast formation, i.e. osteoclastogenesis. See D. E. Hughes et al., Bone, vol. 20, no. 4 (Supp.), April 1997, Abstract No. P362, “Involvement of the Mevalonate Pathway in Osteoclast Apoptosis and the Mechanism of Action of Bisphosphonates”; S. P. Luckman et al., Bone, vol. 20, no. 4 (Supp.), April 1997, Abstract No. P378 “Bisphosphonates and Mevastatin Induce Apoptosis in J774 Macrophages by Inhibition of the Mevalonate Pathway”; and S. P. Luckman et al., Journal of Bone and Mineral Research, vol. 12 (Supp. 1), August 1997, Abstract No. P372 “Bisphosphonates Act By Inhibiting Protein Prenylation”; which are all incorporated by reference herein in their entirety. The HMG-CoA reductase inhibitor, mevastatin, was reported to inhibit in vitro osteoclastogenesis formation in bone marrow cultures. It was also reported that this inhibitory effect was partially restored by the addition of mevalonic acid, a metabolite in the cholesterol biosynthetic pathway. However, it has not been demonstrated in any of these references that the administration of a HMG-CoA reductase inhibitor can actually provide a meaningfully significant therapeutic effect in treating bone resorption and the conditions and disease states associated therewith.

The HMG-CoA reductase inhibitors belong to a class of cardiovascular drugs known as anticholesterolemics. Recent studies have unequivocally demonstrated that lovastatin, simvastatin, and pravastatin, which are all members of the HMG-CoA reductase inhibitor class, slow the progression of atherosclerotic lesions in the coronary and carotid arteries. Simvastatin and pravastatin have also been shown to reduce the risk of coronary heart disease events, and in the case of simvastatin, a highly significant reduction in the risk of coronary death and total mortality has been shown by the Scandinavian Simvastatin Survival Study. However, the use of HMG-CoA reductase inhibitors for treating abnormal bone resorption in humans and other mammals is unknown.

Therefore, the present invention provides novel methods of treatment of abnormal bone resorption comprising administering a therapeutically effective amount of an HMG-CoA reductase inhibitor to a mammal in need thereof. The HMG-CoA reductase inhibitors represent a new class of drugs for treating disorders associated with abnormal bone resorption.

It is therefore an object of the present invention to provide methods for treating abnormal bone resorption and the conditions associated therewith comprising administering a therapeutically effective amount of a HMG-CoA reductase inhibitor to a mammal in need thereof.

It is another object of the present invention to provide methods for treating or preventing, osteoporosis, Paget's disease, periprosthetic bone loss or osteolysis, hypercalcemia of malignancy, osteogenesis imperfecta, osteoarthritis, aveolar bone loss associated with periodontal disease, and abnormal bone resorption associated with immunosuppresive therapy or chronic glucocorticoid use, comprising administering a therapeutically effective amount of a HMG-CoA reductase inhibitor to a mammal in need thereof.

It is another object of the present invention to provide pharmaceutical compositions useful for treating abnormal bone resorption comprising a therapeutically effective amount of a HMG-CoA reductase inhibitor.

It is another object of the present invention to provide methods for treating abnormal bone resorption and the conditions associated therewith by administering a therapeutically effective amount of the combination of a HMG-CoA reductase inhibitor and one or more active agents selected from the group consisting of organic bisphosphonates, estrogen receptor modulators, and peptide hormones, to a mammal in need thereof.

It is another object of the present invention to provide methods for treating or preventing, osteoporosis, Paget's disease, periprosthetic bone loss or osteolysis, hypercalcemia of malignancy, osteogenesis imperfecta, osteoarthritis, aveolar bone loss associated with periodontal disease, and abnormal bone resorption associated with immunosuppresive therapy or chronic glucocorticoid use, comprising administering a therapeutically effective amount of the combination of a HMG-CoA reductase inhibitor and one or more active agents selected from the group consisting of organic bisphosphonates, estrogen receptor modulators, and peptide hormones, to a mammal in need thereof.

It is another object of the present invention to provide pharmaceutical compositions useful for treating abnormal bone resorption comprising a therapeutically effective amount of the combination of a HMG-CoA reductase inhibitor and one or more active agents selected from the group consisting of organic bisphosphonates, estrogen receptor modulators, and peptide hormones, to a mammal in need thereof.

These and other objects will become readily apparent from the detailed description which follows.

### SUMMARY OF THE INVENTION

The present invention relates to a method of inhibiting abnormal bone resorption comprising administering a therapeutically effective amount of a HMG-CoA reductase inhibitor to a mammal in need thereof.

In further embodiments, the present invention relates to a method of inhibiting abnormal bone resorption comprising administering a therapeutically effective amount of the combination of a HMG-CoA reductase inhibitor and one or more active agents selected from the group consisting of organic bisphosphonates, estrogen receptor modulators, and peptide hormones, to a mammal in need thereof.

In further embodiments, the present invention relates to a method of treating or preventing a disease state involving abnormal bone resorption.

In further embodiments, the present invention relates to a pharmaceutical composition comprising a therapeutically effective amount of the combination of an HMG-CoA reductase inhibitor and one or more active agents selected from the group consisting of organic bisphosphonates, estrogen receptor modulators, and peptide hormones.

The invention hereof can comprise, consist of, or consist essentially of the essential as well as optional ingredients, components, and methods described herein

### BRIEF DESCRIPTION OF THE FIGURE

FIG. 1 shows the inhibition of osteoclastogenesis by lovastatin (“lova”, 1 and 10 μM) and its reversal by D,L-mevalonic acid lactone (“MVA”, 1 mM) as determined using a tartrate resistant acid phosphatase fluorescence assay. Osteoclastogenesis is assessed using a co-culture of mouse bone marrow cells and MB 1.8 mouse osteoblasts. The lovastatin and D,L-mevalonic acid lactone are added to the co-cultures at days 5 and 6. Tartrate resistant acid phosphatase activity is measured on day 7. Treatments indicated below each bar graph are as follows: A. no treatment, B. 1 μM lovastatin, C. 1 μM lovastatin and 1 mM D,L-mevalonic acid lactone, D. 10 μM lovastatin, and E. 10 μM lovastatin and 1 mM D,L-mevalonic acid lactone. Results are reported as percent activity relative to no treatment. The error bars indicate the standard error of the mean. The statistical significance of p<0.001 for D and E is determined by Fisher PLSD.

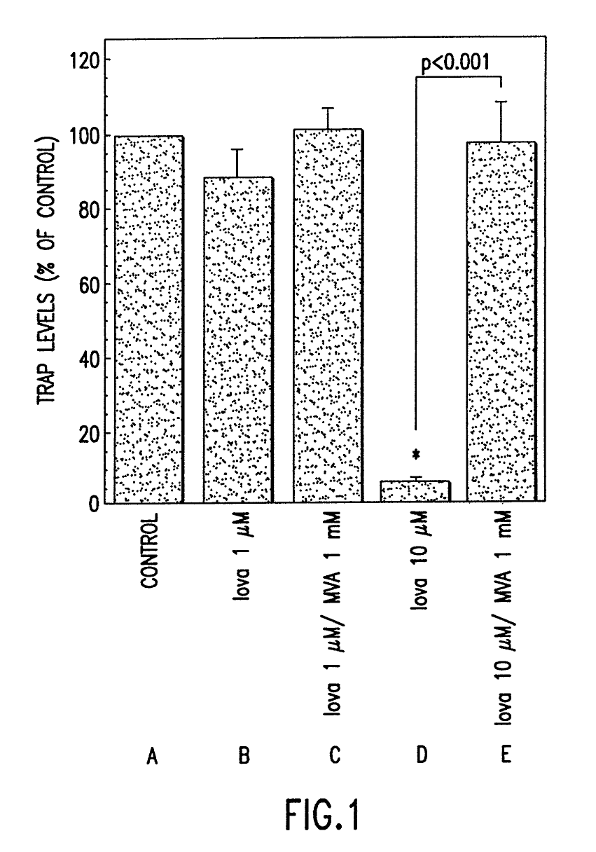

### DETAILED DESCRIPTION OF THE INVENTION

The present invention relates to methods of inhibiting abnormal bone resorption comprising administering a therapeutically effective amount of a HMG-CoA reductase inhibitor to a mammal in need thereof. The methods of the present invention are useful for treating or preventing disease states involving abnormal bone resorption. Typically, the therapeutic regimen of the present invention is administered until the desired therapeutic effect is achieved.

The therapeutic agent useful in the present invention is a compound which inhibits HMG-CoA reductase. Compounds which have inhibitory activity for HMG-CoA reductase can be readily identified by using assays well-known in the art. See U.S. Pat. No. 4,231,938, to Monoghan et al., issued Nov. 4, 1980 and U.S. Pat. No. 5,354,772, to Kathawal, issued Oct. 11, 1994, both of which are incorporated by reference herein in their entirety.

Examples of HMG-CoA reductase inhibitors that are useful herein include but are not limited to lovastatin (MEVACOR®; see U.S. Pat. No. 4,231,938, already cited above and incorporated by reference herein), simvastatin (ZOCOR®; see U.S. Pat. No. 4,444,784, to Hoffman et al., issued Apr. 24, 1984), pravastatin (PRAVACHOL®; see U.S. Pat. No. 4,346,227, to Terahara et al., issued Aug. 24, 1982), fluvastatin (LESCOL®; see U.S. Pat. No. 5,354,772, already cited above and incorporated by reference herein), atorvastatin (LIPITOR®; see U.S. Pat. No. 5,273,995, to Roth, issued Dec. 28, 1993) and cerivastatin (also known as rivastatin; see U.S. Pat. No. 5,177,080, to Angerbauer et al., issued Jan. 5, 1993); and mevastatin (compactin, see U.S. Pat. No. 3,983,140, to Endo et al, issued Sep. 28, 1976. The patents cited in the previous sentence not already incorporated by reference are also incorporated by reference herein in their entirety. The structural formulas of these and additional HMG-CoA reductase inhibitors that can be used in the present invention are described at page 87 of M. Yalpani, “Cholesterol Lowering Drugs”, Chemistry & Industry, pp. 85-89 (5 February 1996), which is incorporated by reference herein in its entirety. The term HMG-CoA reductase inhibitor is intended to include all pharmaceutically acceptable salts, esters and lactone forms of compounds which have HMG-CoA reductase inhibitory activity, and therefor the use of such salts, esters' and lactone forms is included within the scope of this invention. Preferably, the HMG-CoA reductase inhibitor is selected from the group consisting of lovastatin, simvastatin, pravastatin, fluvastatin, atorvastatin, cerivastsin, mevastatin, and the pharmaceutically acceptable salts, esters, and lactones thereof, and mixtures thereof. More preferably, the HMG-CoA reductase inhibitor is selected from the group consisting of lovastatin, simvastatin, pravastatin, fluvastatin, atorvastatin, cerivastsin, and the pharmaceutically acceptable salts, esters, and lactones thereof, and mixtures thereof. More preferably, the HMG-CoA reductase inhibitor is selected from the group consisting of lovastatin, simvastatin, and the pharmaceutically acceptable salts, esters, and lactones thereof, and mixtures thereof.

Preferred HMG-CoA reductase inhibitors can be represented by the chemical formula

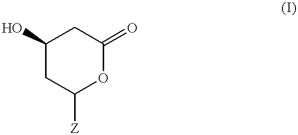

wherein Z is selected from the group consisting of:

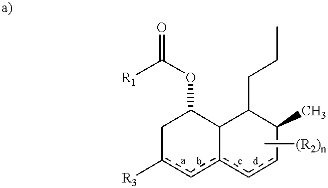

wherein R¹ is C₁-C₁₀ alkyl,

R² is selected from the group consisting of C₁-C₃ alkyl, hydroxy, oxo, and C₁-C₃ hydroxy substituted alkyl,

R³ is selected from the group consisting of hydrogen, hydroxy, C₁-C₃ alkyl, and C₁-C₃ hydroxy substituted alkyl, a, b, c, and d are all single bonds, or a and c are double bonds, or b and d are double bonds, or one of a, b, c, and d is a double bond, and n is 0, 1, or 2;

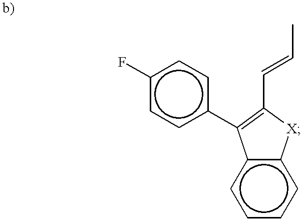

wherein X is selected from the group consisting of N[CH(CH₃)₂] and CH(CH₂)₃CH₃

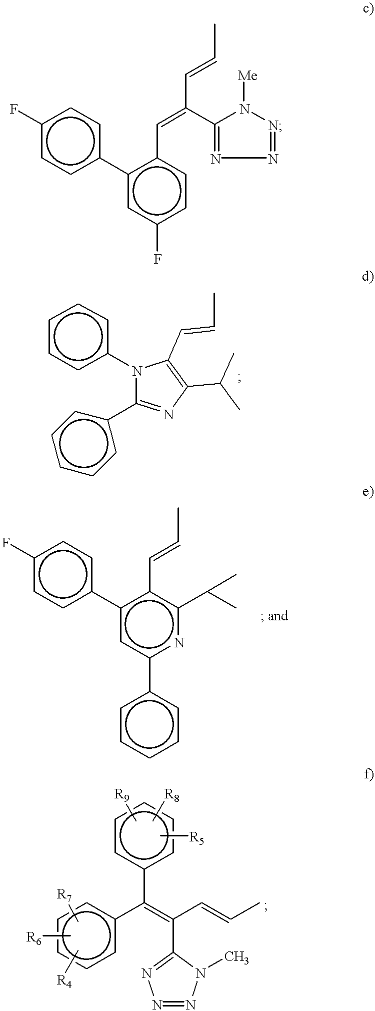

wherein R⁴ and R⁵ are each independently selected from the group consisting of hydrogen, fluorine, chlorine, bromine, iodine, C₁-C₄ alkyl, C₁-C₄ alkoxy, and trifluoromethyl, and R₆, R₇, R₈, and R₉ are each independently selected from the group consisting of hydrogen, fluorine, chlorine, bromine, iodine, C₁-C₄ alkyl, and C₁-C₄ alkoxy. See U.S. Pat. No. 5,650,523, to DeCamp et al., issued Jul. 22, 1997, which is incorporated by reference herein in its entirety. The pharmaceutically acceptable salts, esters, and lactone forms of the compounds depicted by the preceding chemical formulas are intended to be within the scope of the present invention.

The term “pharmaceutically acceptable salts” as used herein in referring to the HMG-CoA reductase inhibitors shall mean non-toxic salts of the compounds employed in this invention which are generally prepared by reacting the free acid with a suitable organic or inorganic base. Examples of salt forms of HMG-CoA reductase inhibitors include, but are not limited to, acetate, benzenesulfonate, benzoate, bicarbonate, bisulfate, bitartrate, borate, bromide, calcium, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynapthoate, iodide, isothionate, lactate, lactobionate, laurate, malate, maleate, mandelate, mesylate, methylbromide, methylnitrate, methylsulfate, mucate, napsylate, nitrate, oleate, oxalate, pamaote, palmitate, panthothenate, phosphate/diphosphate, polygalacturonate, potassium, salicylate, sodium, stearate, subacetate, succinate, tannate, tartrate, teoclate, tosylate, triethiodide, valerate, and mixtures thereof.

The term “esters” as used herein in referring to the HMG-CoA reductase inhibitors is used in its standard meaning to denote the condensation product of a carboxylic acid and an alcohol. Ester derivatives of the described compounds can function as prodrugs which, when absorbed into the bloodstream of a warm-blooded animal, can cleave in such a manner as to release the drug form and permit the drug to afford improved therapeutic efficacy.

The term “lactones” is used herein in referring to the HMG-CoA reductase inhibitors is used in its standard meaning to denote a cyclic condensation product of a carboxylic acid and an alcohol, i.e. a cyclic ester.

The term “therapeutically effective amount”, as used herein, means that amount of the HMG-CoA reductase inhibitor or that amount of the combination of an HMG-CoA reductase inhibitor and one or more active agents that will elicit the desired biological or medical effect or response sought by a medical doctor, clinician, veterinarian, researcher, or other appropriate professional, when administered in accordance with the desired treatment regimen. A preferred therapeutically effective amount is a bone resorption inhibiting amount. The term “therapeutically effective amount” is also intended to encompass prophylactically effective amounts, i.e. amounts that are suitable for preventing a disease state or condition, if a prophylactic or prevention benefit is desired.

The term “abnormal bone resorption”, as used herein means a degree of bone resorption that exceeds the degree of bone formation, either locally, or in the skeleton as a whole. Alternatively, “abnormal bone resorption” can be associated with the formation of bone having an abnormal structure.

The term “bone resorption inhibiting”, as used herein, means preventing bone resorption by the direct or indirect alteration of osteoclast formation or activity. Inhibition of bone resorption refers to prevention of bone loss, especially the inhibition of removal of existing bone either from the mineral phase and/or the organic matrix phase, through direct or indirect alteration of osteoclast formation or activity.

The term “bone resorption inhibiting amount”, as used herein, means that amount of the HMG-CoA reductase inhibitor that will elicit a bone resorption inhibiting effect.

The term “until the desired therapeutic effect is achieved”, as used herein, means that the HMG-CoA reductase inhibitor or the combination with another active agent is administered, according to the dosing schedule chosen, up to the time that the clinical or medical effect sought for the disease or condition being treated or prevented is observed by the clinician or researcher. For the methods of treatment of the present invention, the HMG-CoA reductase inhibitor compound or combination is continuously administered until the desired change in bone mass or structure is observed. In such instances, achieving an increase in bone mass or a replacement of abnormal bone structure with normal bone structure are the desired objectives. For methods of prevention of the present invention, the HMG-CoA reductase inhibitor compound or combination is continuously administered for as long as necessary to prevent the undesired condition or disease state. In such instances, maintenance of bone mass density is often the objective. Nonlimiting examples of treatment and prevention administration periods can range from about 2 weeks to the remaining lifespan of the mammal. For humans, administration periods can range from about 2 weeks to the remaining lifespan of the human, preferably from about 2 weeks to about 20 years, more preferably from about 1 month to about 20 years, more preferably from about 6 months to about 10 years, and most preferably from about 1 year to about 10 years.

The dosage regimen utilizing a HMG-CoA reductase inhibitor or the combination with another active agent is selected in accordance with a variety of factors including type, species, age, weight, sex and medical condition of the patient; the severity of the condition to be treated; the route of administration; the renal and hepatic function of the patient; and the particular compound or salt or ester thereof employed. A consideration of these factors is well within the purview of the ordinarily skilled clinician for the purpose of determining the therapeutically effective or prophylactically effective dosage amounts needed to prevent, counter, or arrest the progress of the condition. The term “patient” includes mammals, especially humans, who take an HMG-CoA reductase inhibitor or combination for any of the uses described herein. Administering of the drug or drugs to the patient includes both self-administration and administration to the patient by another person.

The precise dosage of the HMG-CoA reductase inhibitor or the combination with another active agent will vary with the dosing schedule, the particular compound chosen, the age, size, sex and condition of the mammal or human, the nature and severity of the disorder to be treated, and other relevant medical and physical factors. Thus, a precise pharmaceutically effective amount cannot be specified in advance and can be readily determined by the caregiver or clinician. Appropriate amounts can be determined by routine experimentation from animal models and human clinical studies.

In particular, for daily dosing, the amounts of the HMG-CoA reductase inhibitor can be the same or similar to those amounts which are employed for anti-hypercholesterolemic treatment and which are described in the Physicians' Desk Reference (PDR), 52nd Ed. of the PDR, 1998 (Medical Economics Co), which is incorporated by reference herein in its entirety. For the additional active agents, the doses can be the same or similar to those amounts which are known in the art.

The HMG-CoA reductase inhibitors and the combination with other active agents can be administered via a wide variety of routes including oral administration, intravenous administration, intranasal administration, injections, ocular administration, and the like.

A preferred route of delivery is oral administration.

Oral dosage amounts of the HMG-CoA reductase inhibitor are from about 1 to 200 mg/day, and more preferably from about 5 to 160 mg/day. However, dosage amounts will vary depending on the potency of the specific HMG-CoA reductase inhibitor used as well as other factors as noted above. An HMG-CoA reductase inhibitor which has sufficiently greater potency may be given in sub-milligram daily dosages. The HMG-CoA reductase inhibitor may be administered from 1 to 4 times per day, and preferably once per day.

For example, the daily dosage amount for simvastatin can be selected from 5 mg, 10 mg, 20 mg, 40 mg, 80 mg and 160 mg; for lovastatin, 10 mg, 20 mg, 40 mg and 80 mg; for fluvastatin sodium, 20 mg, 40 mg and 80 mg; for pravastatin sodium, 10 mg, 20 mg, and 40 mg; and for atorvastatin calcium, 10 mg, 20 mg, and 40 mg.

Additional Active Agents For Inhibiting Abnormal Bone Resorption

Further exemplifying the invention are methods of treatment comprising administering a HMG-CoA reductase inhibitor in combination with one or more active agents for inhibiting bone abnormal resorption selected from the group consisting of organic bisphosphonates, estrogen receptor modulators, and peptide hormones.

These additional active agents for inhibiting bone resorption can be used in combination with the HMG-CoA reductase inhibitor in a single dosage formulation, or may be administered to the patient in a separate dosage formulation, which allows for concurrent or sequential administration.

Organic Bisphosohonates

The bisphosphonates useful herein correspond to the chemical formula

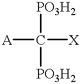

wherein

A and X are independently selected from the group consisting of H, OH, halogen, NH₂, SH, phenyl, C1-C30 alkyl, C1-C30 substituted alkyl, C1-C10 alkyl or dialkyl substituted NH₂, C1-C10 alkoxy, C1-C10 alkyl or phenyl substituted thio, C1-C10 alkyl substituted phenyl, pyridyl, furanyl, pyrrolidinyl, imidazonyl, and benzyl.

In the foregoing chemical formula, the alkyl groups can be straight, branched, or cyclic, provided sufficient atoms are selected for the chemical formula. The C1-C30 substituted alkyl can include a wide variety of substituents, nonlimiting examples which include those selected from the group consisting of phenyl, pyridyl, furanyl, pyrrolidinyl, imidazonyl, NH₂, C1-C10 alkyl or dialkyl substituted NH₂, OH, SH, and C1-C10 alkoxy.

In the foregoing chemical formula, A can include X and X can include A such that the two moieties can form part of the same cyclic structure.

The foregoing chemical formula is also intended to encompass complex carbocyclic, aromatic and hetero atom structures for the A and/or X substituents, nonlimiting examples of which include naphthyl, quinolyl, isoquinolyl, adamantyl, and chlorophenylthio.

Preferred structures are those in which A is selected from the group consisting of H, OH, and halogen, and X is selected from the group consisting of C1-C30 alkyl, C1-C30 substituted alkyl, halogen, and C1-C10 alkyl or phenyl substituted thio.

More preferred structures are those in which A is selected from the group consisting of H, OH, and Cl, and X is selected from the group consisting of C1-C30 alkyl, C1-C30 substituted alkyl, Cl, and chlorophenylthio.

Most preferred is when A is OH and X is a 3-aminopropyl moiety, so that the resulting compound is a 4-amino-1-hydroxybutylidene-1,1-bisphosphonate, i.e. alendronate.

Pharmaceutically acceptable salts and derivatives of the bisphosphonates are also useful herein. Nonlimiting examples of salts include those selected from the group consisting of alkali metal, alkaline metal, ammonium, and mono-, di, tri-, or tetra-C1-C30-alkyl-substituted ammonium. Preferred salts are those selected from the group consisting of sodium, potassium, calcium, magnesium, and ammonium salts. Nonlimiting examples of derivatives include those selected from the group consisting of esters, hydrates, and amides.

“Pharmaceutically acceptable” as used herein means that the salts and derivatives of the bisphosphonates have the same general pharmacological properties as the free acid form from which they are derived and are acceptable from a toxicity viewpoint.

It should be noted that the terms “bisphosphonate” and “bisphosphonates”, as used herein in referring to the therapeutic agents of the present invention are meant to also encompass diphosphonates, biphosphonic acids, and diphosphonic acids, as well as salts and derivatives of these materials. The use of a specific nomenclature in referring to the bisphosphonate or bisphosphonates is not meant to limit the scope of the present invention, unless specifically indicated. Because of the mixed nomenclature currently in use by those or ordinary skill in the art, reference to a specific weight or percentage of a bisphosphonate compound in the present invention is on an acid active weight basis, unless indicated otherwise herein. For example, the phrase “about 70 mg of a bone resorption inhibiting bisphosphonate selected from the group consisting of alendronate, pharmaceutically acceptable salts thereof, and mixtures thereof, on an alendronic acid active weight basis” means that the amount of the bisphosphonate compound selected is calculated based on 70 mg of alendronic acid.

Nonlimiting examples of bisphosphonates useful herein include the following:

Alendronic acid, 4-amino-1-hydroxybutylidene-1,1-bisphosphonic acid.

Alendronate (also known as alendronate sodium or monosodium trihydrate), 4-amino-1-hydroxybutylidene-1,1-bisphosphonic acid monosodium trihydrate.

Alendronic acid and alendronate are described in U.S. Pat. Nos. 4,922,007, to Kieczykowski et al., issued May 1, 1990, and 5,019,651, to Kieczykowski, issued May 28, 1991, both of which are incorporated by reference herein in their entirety.

Cycloheptylaminomethylene-1,1-bisphosphonic acid, YM 175, Yamanouchi (cimadronate), as described in U.S. Pat. No. 4,970,335, to Isomura et al., issued Nov. 13, 1990, which is incorporated by reference herein in its entirety.

1,1-dichloromethylene-1,1-diphosphonic acid (clodronic acid), and the disodium salt (clodronate, Procter and Gamble), are described in Belgium Patent 672,205 (1966) and J. Org. Chem 32, 4111 (1967), both of which are incorporated by reference herein in their entirety.

1-hydroxy-3-(1-pyrrolidinyl)-propylidene-1,1-bisphosphonic acid (EB-1053).

1-hydroxyethane-1,1-diphosphonic acid (etidronic acid).

1-hydroxy-3-(N-methyl-N-pentylamino)propylidene-1,1-bisphosphonic acid, also known as BM-210955, Boehringer-Mannheim (ibandronate), is described in U.S. Pat. No. 4,927,814, issued May 22, 1990, which is incorporated by reference herein in its entirety.

6-amino-1-hydroxyhexylidene-1,1-bisphosphonic acid (neridronate).

3-(dimethylamino)-1-hydroxypropylidene-1,1-bisphosphonic acid (olpadronate).

3-amino-1-hydroxypropylidene-1,1-bisphosphonic acid (pamidronate).

[2-(2-pyridinyl)ethylidene]-1,1-bisphosphonic acid (piridronate) is described in U.S. Pat. No. 4,761,406, which is incorporated by reference in its entirety.

1-hydroxy-2-(3-pyridinyl)-ethylidene-1,1-bisphosphonic acid (risedronate).

(4-chlorophenyl)thiomethane-1,1-disphosphonic acid (tiludronate) as described in U.S. Pat. No. 4,876,248, to Breliere et al., Oct. 24, 1989, which is incorporated by reference herein in its entirety.

1-hydroxy-2-(1H-imidazol-1-yl)ethylidene-1,1-bisphosphonic acid (zolendronate).

Preferred are bisphosphonates selected from the group consisting of alendronate, cimadronate, clodronate, tiludronate, etidronate, ibandronate, risedronate, piridronate, paindronate, zolendronate, pharmaceutically acceptable salts thereof, and mixtures thereof.

More preferred is alendronate, pharmaceutically acceptable salts thereof, and mixtures thereof.

Most preferred is alendronate monosodium trihydrate.

Estrogen Receptor Modulators

Estrogen receptor modulators are known for use in hormone replacement therapy and for their anti-bone resorption benefits.

Nonlimiting examples of estrogen receptor modulators useful herein include estrogen, progestins, estradiol, raloxifene, and tamoxifene, and their pharmaceutically acceptable salts, and mixtures thereof.

Peptide Hormones

A peptide hormone useful herein is calcitonin, which is approved for use for treating osteoporosis. Both human and salmon calcitonin are useful herein.

Pharmaceutical Compositions

Compositions useful in the present invention comprise a therapeutically effective amount of a HMG-CoA reductase inhibitor. In further embodiments, these compositions also comprise one or more active agents. The HMG-CoA reductase inhibitor and any other active agents is typically administered in admixture with suitable pharmaceutical diluents, excipients, or carriers, collectively referred to herein as “carrier materials”, selected with respect to the route of administration, i.e. for example oral administration, intravenous administration, intranasal administration, injection, or ocular administration.

For oral administration, the composition can be administered in the form of tablets, capsules, elixirs, syrups, powders, and the like, and consistent with conventional pharmaceutical practices. For solid oral forms, e.g. tablets, capsules, or powders, the HMG-CoA reductase inhibitor and any other active agents can be combined with an oral, non-toxic, pharmaceutically acceptable inert carrier such as lactose, starch, sucrose, glucose, methyl cellulose, magnesium stearate, mannitol, sorbitol, croscarmellose sodium and the like. For liquid oral forms, e.g., elixirs and syrups, the drug component or components can be combined with any oral, non-toxic, pharmaceutically acceptable inert carrier such as ethanol, glycerol, water and the like. Moreover, when desired or necessary, suitable binders, lubricants, disintegrating agents and coloring agents can also be incorporated. Suitable binders can include starch, gelatin, natural sugars such a glucose, anhydrous lactose, free-flow lactose, beta-lactose, and corn sweeteners, natural and synthetic gums, such as acacia, guar, tragacanth or sodium alginate, carboxymethyl cellulose, polyethylene glycol, waxes, and the like. Lubricants used in these dosage forms include sodium oleate, sodium stearate, magnesium stearate, sodium benzoate, sodium acetate, sodium chloride and the like.

The drug or drugs can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The drug or drugs can also be delivered by the use of monoclonal antibodies as individual carriers to which the compound molecules are coupled. Active drug or drugs can also be coupled with soluble polymers as targetable carriers. Such polymers can include polyvinyl-pyrrolidone, pyran copolymer, polyhydroxy-propyl-methacrylamide-phenol, polyhydroxy-ethyl-aspartamide-phenol, or polyethyleneoxide-polylysine substituted with palmitoyl residues. Furthermore, active drug may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and cross linked or amphipathic block copolymers of hydrogels.

The instant invention includes the use of both rapid-release and time-controlled release pharmaceutical formulations.

Process for Preparing Pharmaceutical Compositions

The instant invention also encompasses a process for preparing a pharmaceutical composition comprising combining the HMG-CoA reductase inhibitor and any other active agents with a pharmaceutically acceptable carrier. The instant invention also encompasses the use of an HMG-CoA reductase inhibitor and any other active agents for the preparation of a medicament for inhibiting abnormal bone resorption.

The following Examples are presented to better illustrate the invention.

### EXAMPLE 1

### Method for Evaluating the Effect of HMG-CoA Reductase Inhibitors on Osteoclastoaenesis in Murine Co-Cultures

Murine co-cultures of osteoblasts and marrow cells are prepared using the methods of Wesolowski, et al., Exp Cell Res, (1995), 219, pp. 679-686, which is incorporated by reference herein in its entirety. Bone marrow cells are harvested from 6-week-old male Balb/C mice by flushing marrow spaces of freshly isolated long bones (tibiae and femora) with α-MEM (minimal essential media) containing penicillin/streptomycin (100 I.U./ml of each and 20 mM Hepes buffer). The bone marrow cells are suspended in α-MEM and the cells are filtered through an approximately 70 μm cell strainer. The filtrate is centrifuged at about 300 x g for about 7 minutes. The resulting pellet is resuspended in α-MEM supplemented with fetal calf serum (10% v/v) and 10 nM 1, 25-(OH)₂ vitamin D₃. These bone marrow isolates are added to sub-confluent monolayers of osteoblastic MB 1.8 cells in 24 well cell culture plates and cultured for 5 days at 37° C. in the presence of 5% CO₂. Culture media is replenished daily. Fusion of the osteoclast precursor cells from bone marrow (with each other) to form multinucleated osteoclast-like cells typically occurs after about 5 days.

The compounds to be evaluated are prepared as a solution of the desired concentration in α-MEM. Examples of compounds evaluated include the HMG-CoA reductase inhibitors, lovastatin and simvastatin, as well as compounds that block the effects of these inhibitors, such as D,L-mevalonic acid lactone. Combinations of compounds can also be evaluated. The solutions of the compounds to be evaluated are added to the cultures, typically about 0.5 mL/well, on days 5 and 6. No treatment controls (controls not treated with compounds) are prepared by adding equivalent volumes of α-MEM to the control wells. On day 7, the cultures are evaluated by counting the number of osteoclast-like cells (stained multinucleated cells) or by measuring the tartrate-resistant acid phosphatase (TRAP) activity of the sample via standard fluorescence techniques using a naphthol-based substrate.

Staining and counting of osteoclast like cells

The following solutions are prepared for staining the cultures:

3.7% formalin: 1:10 dilution of 37% formaldehyde in phosphate-buffered 0.9 % NaCl,

HBS: 0.9% NaCl, 10 mM HEPES, pH 7.1,

Acetate/Tartrate buffer: 50 mM sodium acetate, 30 mM sodium tartrate, 0.1% Triton X-100, pH 5.0,

Staining Solution: dissolve Fast Red Violet LB (Sigma # F1625) in acetate/tartrate buffer at 0.3 mg/ml and add 5 μl/ml of 20 mg/ml solution of Napthol AS-MX phosphate in acetate/tartrate buffer (this solution is made fresh just prior to staining).

The cell cultures are fixed for 10 minutes with approximately 0.5 mL of 3.7% formalin at room temperature and then washed once with about 1 mL of the HBS. The staining solution (about 0.5 mL) is added to each well and the plates are then incubated for about 10-20 minutes at about 37° C. Following staining, each plate is washed 3 times with de-ionized water, blotted on paper towels and then allowed to air dry. Multi-nucleated stained cells are counted using an inverted-phase microscope at about 30x magnification.

Fluorescence measurements

The following substrate solution is prepared for measuring TRAP activity via fluorescence:

Substrate solution: A compound having a napthol functional group is dissolved at a concentration of 2.5 mg/ml in HBS buffer.

The cell cultures are are washed with HBS (about 0.5 ml) and then treated with commercially-available trypsin/EDTA (Gibco BRL, Grand Island, N.Y., 0.25 ml/well) for 10 minutes at 37° C. to selectively release the mononuclear multinuclear cells. Following trypsinization the plates are washed 3 times with Hepes and then blotted on paper towels. Next, about 0.5 mL of the naphthol substrate solution is added to each well and the plates are then incubated at 37° C. Reactions are stopped 1 hour after incubation by addition of 1M NaOH (about 0.05 ml/well). The contents of the wells are swirled by placing the plates on an orbital shaker for about 10 minutes to dissolve any precipitates. Fluorescence is determined using a fluorescence plate reader with the excitation wavelength set at 360 nm and the emission wavelength set at 530 nm.

Using either the visual counting or fluorescence techniques it is demonstrated that HMG-CoA reductase inhibitors inhibit osteoclastogenesis.

### EXAMPLE 2

Tablet composition

| Ingredient | Amount per tablet |
|---|---|
| Simvastatin | 5.0 mg |
| BHA | 0.02 mg |
| Ascorbic acid | 2.50 mg |
| Citric acid | 1.25 mg |
| Microcrystalline cellulose | 5.0 mg |
| Pregel starch | 10.0 mg |
| Magnesium stearate | 0.5 mg |
| Lactose | 74.73 mg |

All the ingredients except magnesium stearate are blended together in a suitable mixer. The powder mixture is then granulated with adequate quantities of granulating solvent(s), e.g. water. The wet granulated mass is dried in a suitable dryer. The dried granulation is sized through a suitable screen. The sized granulation is mixed with magnesium stearate before tableting. The tablets may be coated if deemed necessary. Additional ingredients that may be added to the above include suitable color and mixtures of colors.

The composition is useful for inhibiting abnormal bone resorption.

In alternative formulations, the simvastatin is replaced by an HMG-CoA reductase inhibitor selected from lovastatin, pravastatin, fluvastatin, atorvastatin, cerivastsin, and mevastatin.

### EXAMPLE 3

Directly compressed tablet composition

| Amount per tablet | Ingredient |
|---|---|
| 5 mg | Lovastatin |
| 116.9 mg | Microcrystalline cellulose |
| 116.9 mg | Lactose anhydrate |
| 7.5 mg | Crosmellose sodium |
| 3.7 mg | Magnesium stearate |

The ingredients are combined and blended together and are compressed using conventional tableting techniques.

The composition is useful for inhibiting abnormal bone resorption.

In alternative formulations, the lovastatin is replaced by an HMG-CoA reductase inhibitor selected from lovastatin, pravastatin, fluvastatin, atorvastatin, cerivastsin, and mevastatin.

### EXAMPLE 4

Hard gelatin capsule composition

| Amount per capsule | Ingredient |
|---|---|
| 5 mg | Simvastatin |
| 47 mg | Microcrystalline cellulose |
| 47 mg | Lactose anhydrate |
| 1 mg | Magnesium stearate |
| 1 capsule | Hard gelatin capsule |

The dry ingredients are combined and blended together and encapsulated in a gelatin coating using standard manufacturing techniques.

The composition is useful for inhibiting abnormal bone resorption.

In alternative formulations, the simvastatin is replaced by an HMG-CoA reductase inhibitor selected from lovastatin, pravastatin, fluvastatin, atorvastatin, cerivastsin, and mevastatin.

### EXAMPLE 5

Oral suspension composition

| Amount per 5 mL dose | Ingredient |
|---|---|
| 5 mg | Lovastatin |
| 150 mg | Polyvinylpyrrolidone |
| 2.5 mg | Poly oxyethylene sorbitan monolaurate |
| 10 mg | Benzoic acid |
| to 5 mL with aqueous sorbitol | |
| solution (70%) | |

An oral suspension is prepared by combining the ingredients using standard formulation techniques.

The composition is useful for inhibiting abnormal bone resorption.

In alternative formulations, the lovastatin is replaced by an HMG-CoA reductase inhibitor selected from simvastatin, pravastatin, fluvastatin, atorvastatin, cerivastsin, and mevastatin.

### EXAMPLE 6

Intravenous infusion comnosition

| Amount per 200 mL dose | Ingredient |
|---|---|
| 5 mg | Simvastatin |
| 0.2 mg | Polyethylene oxide 400 |
| 1.8 mg | Sodium chloride |
| to 200 mL | Purified water |

The ingredients are combined using standard formulation techniques.

In alternative formulations, the simvastatin is replaced by an HMG-CoA reductase inhibitor selected from lovastatin, pravastatin, fluvastatin, atorvastatin, cerivastsin, and mevastatin.

What is claimed is:

1. A method of inhibiting abnormal bone resorption comprising administering a therapeutically effective amount of a HMG-CoA reductase inhibitor to a mammal in need thereof.

2. A method according to claim 1 wherein said mammal is a human.

3. A method according to claim 2 wherein said HMG-CoA reductase inhibitor is selected from the group consisting of lovastatin, simvastatin, pravastatin, fluvastatin, atorvastatin, cerivastsin, mevastatin, and the pharmaceutically acceptable salts, esters, and lactones thereof, and mixtures thereof.

4. A method according to claim 3 wherein said HMG-CoA reductase inhibitor is selected from the group consisting of lovastatin, simvastatin, pravastatin, fluvastatin, atorvastatin, cerivastsin, and the pharmaceutically acceptable salts, esters, and lactones thereof, and mixtures thereof.

5. A method according to claim 4 wherein said HMG-CoA reductase inhibitor is selected from the group consisting of lovastatin, simvaststin, and the pharmaceutically acceptable salts, esters, and lactones thereof, and mixtures thereof.

6. A method of inhibiting abnormal bone resorption comprising administering a therapeutically effective amount of the combination of a HMG-CoA reductase inhibitor and one or more active agents selected from the group consisting of organic bisphosphonates, estrogen receptor modulators, and peptide hormones to a mammal in need thereof.

7. A method according to claim 6 wherein said mammal is a human.

8. A method according to claim 7 wherein said HMG-CoA reductase inhibitor is selected from the group consisting of lovastatin, simvastatin, pravastatin, fluvastatin, atorvastatin, cerivastsin, mevastatin, and the pharmaceutically acceptable salts, esters, and lactones thereof, and mixtures thereof.

9. A method according to claim 8 wherein said HMG-CoA reductase inhibitor is selected from the group consisting of lovastatin, simvastatin, pravastatin, fluvastatin, atorvastatin, cerivastsin, and the pharmaceutically acceptable salts, esters, and lactones thereof, and mixtures thereof.

10. A method according to claim 9 wherein said HMG-CoA reductase inhibitor is selected from the group consisting of lovastatin, simvaststin, and the pharmaceutically acceptable salts, esters, and lactones thereof, and mixtures thereof.

11. A method according to claim 7 wherein said organic bisphosphonate is selected from the group consisting of alendronate, cimadronate, clodronate, tiludronate, etidronate, ibandronate, risedronate, piridronate, pamidronate, zolendronate, pharmaceutically acceptable salts thereof, and mixtures thereof.

12. A method according to claim 11 wherein said organic bisphosphonate is alendronate and the pharmaceutically acceptable salts thereof.

13. A method according to claim 12 wherein said organic bisphosphonate is alendronate monosodium trihydrate.

14. A method according to claim 7 wherein said estrogen receptor modulator is selected from the group consisting of estrogen, progestins, estradiol, raloxifene, and tamoxifene, and their pharmaceutically acceptable salts, and mixtures thereof.

15. A method according to claim 7 wherein said peptide hormone is selected from the group consisting of human calcitonin, salmon calcitonin, and mixtures thereof.

16. A pharmaceutical composition comprising a therapeutically effective amount of the combination of a HMG-CoA reductase inhibitor and one or more active agents selected from the group consisting of organic bisphosphonates, estrogen receptor modulators, and peptide hormone.

17. A composition according to claim 16 which further comprises a pharmaceutically acceptable carrier.

18. A composition according to claim 17 wherein said HMG-CoA reductase inhibitor is selected from the group consisting of lovastatin, simvastatin, pravastatin, fluvastatin, atorvastatin, cerivastsin, mevastatin, and the pharmaceutically acceptable salts, esters, and lactones thereof, and mixtures thereof.

19. A composition according to claim 17 wherein said organic bisphosphonate is selected from the group consisting of alendronate, cimadronate, clodronate, tiludronate, etidronate, ibandronate, risedronate, piridronate, pamidronate, zolendronate, pharmaceutically acceptable salts thereof, and mixtures thereof.

20. A composition according to claim 17 wherein said estrogen receptor modulator is selected from the group consisting of estrogen, progestins, estradiol, raloxifene, and tamoxifene, and their pharmaceutically acceptable salts, and mixtures thereof.

21. A composition according to claim 17 wherein said peptide hormone is selected from the group consisting of human calcitonin, salmon calcitonin, and mixtures thereof.

22. A method of treating or preventing a disease state involving abnormal bone resorption comprising administering a therapeutically effective amount of a HMG-CoA reductase inhibitor to a mammal in need thereof.

23. A method according to claim 22 wherein said disease state is osteoporosis.

24. A method of treating or preventing a disease state involving abnormal bone resorption comprising administering a therapeutically effective amount of the combination of a HMG-CoA reductase inhibitor and one or more active agents selected from the group consisting of organic bisphosphonates, estrogen receptor modulators, and peptide hormones to a mammal in need thereof.

25. A method according to claim 24 wherein said disease state is osteoporosis.
